(19) United States
(12) Patent Application Publication
(10) Pub. No.: US 2009/0101577 A1
(43) Pub. Date: Apr. 23, 2009

(54) Methods and Systems for Controlling Ultrafiltration Using Central Venous Pressure Measurements

(76) Inventors: Barry N. Fulkerson, Longmont, CO (US); James R. Braig, Piedmont, CA (US); Victor Gura, Los Angeles, CA (US)

Correspondence Address: PATENTMETRIX, 14252 CULVER DR. BOX 914, IRVINE, CA, 92604, US

(21) Appl. No.: 12/238,055

(22) Filed: Sep. 25, 2008

Related U.S. Application Data
(60) Provisional application No. 60/975,840, filed on Sep. 28, 2007.

(51) Int. Cl. B01D 61/34; A61B 5/02

(52) U.S. Cl. 210/646; 600/485

(57) ABSTRACT

The volume of fluid removed from a patient during ultrafiltration is controlled automatically on the basis of central venous pressure (CVP) measurements. In one embodiment, a central venous catheter (CVC) is used for accessing blood during dialysis. A sensor located at the tip of the catheter or inside the dialysis machine is used to periodically measure CVP. CVP feedback data helps prevent the excessive removal of fluids from the patient.

### CROSS-REFERENCE

The present application relies on U.S. Patent Provisional Application No. 60/975,840 filed on Sep. 28, 2007 for priority.

### FIELD OF THE INVENTION

The present invention relates generally to the field of blood purification systems and methods. More specifically, the present invention is directed to monitoring and controlling ultrafiltration using central venous pressure. It is further directed to systems for measuring central venous pressure that are integrated with dialysis systems.

### BACKGROUND OF THE INVENTION

When performing ultrafiltration (UF) for patients suffering from renal impairment, excess fluid from the patient's blood is removed by extracting the blood into an excorporeal device that passes the blood across a semi permeable membrane, having a pressure gradient applied thereto. The semi permeable membrane is embodied as a highly permeable hemofilter or dialyzer. One of the potential risks to a patient's health associated with the UF procedure is hypotension, which is an abnormal decrease in the patient's blood pressure. An abnormally high or uncontrolled ultrafiltration rate may result in hypovolemic shock, hypotension, or both. This happens when too much water is removed from the patient's blood, as a result of the ultrafiltration rate being too high or uncontrolled.

Ideally, during UF treatment, plasma volume in the patient's blood should remain constant. Volume consistency can occur if the plasma refilling rate and fluid recovered from interstitial spaces equals the UF removal rate. However, refilling of the plasma is often not completed at the appropriate rate, thereby leading to excessive volume loss or, at the least, excessively rapid volume loss.

Long term loss of blood volume can lead to reduced cardiac output, which, in turn, decreases renal blood flow, eventually leading to renal failure. While some UF devices may be able to remove the right amount of fluid pursuant to a specific removal rate, conventional UF devices cannot effectively adapt to a patient's needs. Conventional UF devices therefore require constant attention and monitoring by a health care provider, thereby making them unsuitable for home or other unattended use.

Certain UF monitoring approaches have been proposed in the art. For example, hemoglobin oxygen saturation level has been proposed as an indicator of impending hypotension. U.S. Patent Application No. US20020085951A1 discloses the use of oxygen saturation level of the venous blood (SvO2) as an early indicator of hypotension. A sharp fall in SvO2 can be used as an indicator to reduce or cease hemofiltration.

U.S. Pat. No. 7,115,095 discloses the use of average left atrial (LAP) pressure as an indicator of hypotension. While not targeted at UF, this patent discloses a method for using a pressure monitor to indicate the need for treating hypotension. LAP is typically 12 mmHg in a normal individual; patients with fluid overload caused by congestive heart failure have LAP pressures above 15-20 mmHg. Knowing the pressure allows medication, typically diuretics, to be used to remove excess fluid and return the pressure to normal.

Central venous pressure (CVP) is useful for assessing the volume status of the patient. CVP can be used to guide fluid therapy in a patient with hypovolaemia following trauma, shock, burns, or sepsis. CVP catheters can be inserted at different sites, but, in each case, the tip of the catheter should be intrathoracic. Sites used for the insertion of cannulae include the external jugular vein, internal jugular vein (high or low approach), subclavian vein, femoral vein and the antecubital vein. Normal CVP is 2-6 mmHg. Elevated CVP is indicative of over hydration, while decreased CVP indicates hypovolaemia.

U.S. Pat. No. 6,471,872 discloses the monitoring of 13 different patient variables and the use of some of these variables to control dialysis. While CVP is disclosed as being monitored, there is no disclosure of how such monitoring occurs or whether (or how) such CVP measurement can be integrated into a dialysis process. Rather, the patient appears to require CVP measurement to be done outside the dialysis system and electronically communicated to the dialysis system, without possibility of measuring CVP using the pre-existing connections of the dialysis system.

U.S. Pat. No. 7,175,809 discloses the regulation of fluid removal in hemofiltration by monitoring oxygen level in venous blood and teaches that a sudden drop in SvO2 indicates impending hypotension and fluid removal should be curtailed. While the patent does teach that if CVP drops hypotension may ensue, the patent does not teach any means to measure CVP or the measurement of CVP integrated with dialysis.

U.S. Pat. No. 6,623,470 discloses the delivery of fluids and the monitoring of venous pressure to indicate adequate delivery of fluids. In the event of a pending major blood loss such as in surgery, Central Venous Pressure (CVP) is monitored as fluid is infused to maintain CVP at normal levels.

Thus, there is no satisfactory mechanism in the prior art for continually monitoring and controlling the volume of fluid in a patient during dialysis/ultrafiltration. Therefore, there is a need to have a means for effectively controlling fluid volume and the UF rate during such procedures. It would also be desirable to have a system that integrates CVP measurement into the dialysis/ultrafiltration system itself. It would be further preferable to have a system where UF rate can be controlled to be limited within a range, based on CVP measurement and monitoring.

### SUMMARY OF THE INVENTION

The present application is directed toward, in one embodiment, a method for regulating the volume of fluid removed from a patient during renal dialysis by periodically measuring the average central venous pressure in the ventral venous line used for dialysis; and adjusting the rate of ultrafiltration based on the measured values of central venous pressure. Optionally, the frequency of central venous pressure measurement and an acceptable range of central venous pressure values is preset. Ultrafiltration is discontinued when central venous pressure drops below a preset limit. The rate of ultrafiltration and/or total volume of fluid to be removed from said patient is preset. The flow of blood when central venous pressure is measured is stopped. The method can be used with any one of a hemofiltration system, a hemodiafiltration system, or a hemodialysis system.

The present application is also directed to a system for regulating the volume of fluid removed from a patient during renal dialysis, the system comprising a sensor for periodically measuring the average central venous pressure in the ventral venous line used for dialysis; and a controller for causing said sensor to periodically measure the average central venous pressure and adjusting the rate of ultrafiltration based on the measured values of central venous pressure.

Optionally, the controller is programmable to operate according to a preset frequency of central venous pressure measurement and a preset acceptable range of central venous pressure values. The controller is programmable to operate according to a preset rate of ultrafiltration. The controller is programmable to operate according to a preset total volume of fluid that is to be removed from said patient. The controller is configured to discontinue ultrafiltration when central venous pressure drops below a preset limit. The controller stops the flow of blood when central venous pressure is measured. The sensor for measuring central venous pressure is located at the tip of a catheter used for accessing blood during dialysis. The sensor for measuring central venous pressure is located remote from the catheter used for accessing blood during dialysis. The sensor for measuring central venous pressure is located at the same level as the heart. The sensor for measuring central venous pressure is located inside the dialysis machine. The catheter used for accessing blood during dialysis is a central venous catheter. The central venous catheter is a double lumen catheter. The system is used with any one of a hemofiltration system, a hemodiafiltration system, or a hemodialysis system.

### BRIEF DESCRIPTION OF THE DRAWINGS

These and other features and advantages of the present invention will be appreciated, as they become better understood by reference to the following detailed description when considered in connection with the accompanying drawings, wherein:

FIG. 1 depicts an exemplary location of a central venous catheter for hemofiltration;

FIG. 2 depicts an exemplary location of a catheter for CVP monitoring;

FIG. 3 is a block diagram illustrating the programmable controller as used in the present invention; and

FIG. 4 depicts an exemplary blood circuit of the present invention.

### DETAIL DESCRIPTION OF THE INVENTION

The present invention is directed towards novel methods and systems for monitoring and controlling the UF rate, such that the volume of fluid within a patient undergoing dialysis/ultrafiltration remains within a desired range. This invention integrates central venous pressure (CVP) monitoring into a dialysis system and uses CVP measurements to control the rate of ultrafiltration (UF). CVP feedback data helps prevent over removal of fluids as a safety measure and provides a means for titrating the UF rate for improving therapy.

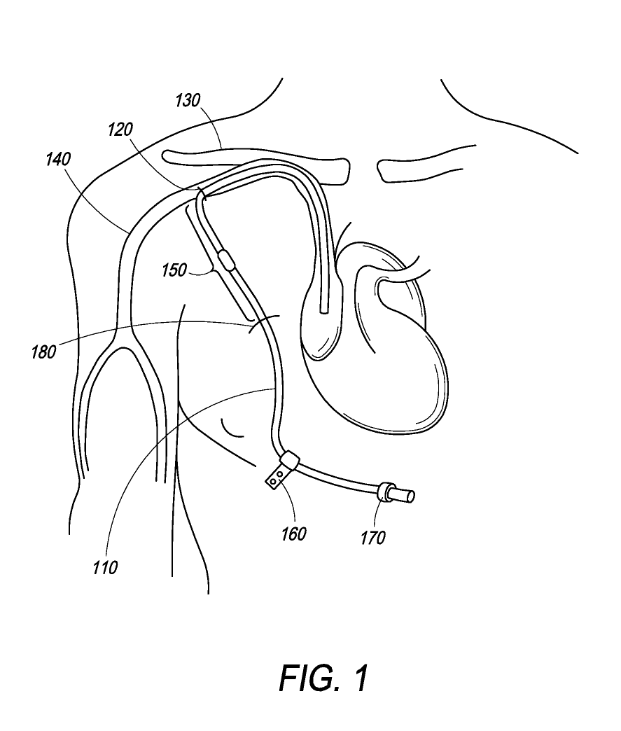

The present invention entails measuring the average pressure present in the ventral venous line used for dialysis, thereby integrating CVP measurement with dialysis. In order to measure CVP, an appropriate catheter needs to be inserted in the patient's body, such that the tip of the catheter is placed intrathoracically. FIG. 1 depicts an exemplary location of a central venous catheter for hemofiltration and CVP measurement. Referring to FIG. 1, a Central Venous Catheter (CVC) 110 is used to provide vascular access for UF. In this particular embodiment, the entrance site 120 chosen for the CVC 110 is below the collarbone (clavicle) 130, at the subclavian vein 140. One of ordinary skill in the art would appreciate that any other large vein in the patient's body may be selected as an alternate site for inserting the CVC, while keeping its tip intrathoracic. The CVC 110 passes through a subcutaneous tunnel 150, and is secured with the help of a clamp 160 and a standard leur-lock 170. Pressure at the tip of the CVC at the exit site 180 is equal to the Central Venous Pressure.

In one embodiment of the present invention, the CVC 110 is used for accessing blood during hemofiltration, and the central venous pressure may be measured using sensors inside the hemofiltration machine. In this case, no additional equipment is required for CVP measurement. In another embodiment, a dual lumen CVC is used for hemofiltration. In this case, the proximal lumen can be used for blood withdrawal and the distal lumen (at the tip) can be used for returning blood. Either lumen or port can provide a CVP measurement. In both cases, when a CVC is used for blood access, the system of present invention provides that prior to taking a CVP measurement, blood flow is momentarily stopped to enable the accurate measurement of pressure. Therefore, in one embodiment, the present invention integrates into conventional dialysis machines programmatic controls for stopping blood flow through the device based upon a predetermined CVP measurement rate.

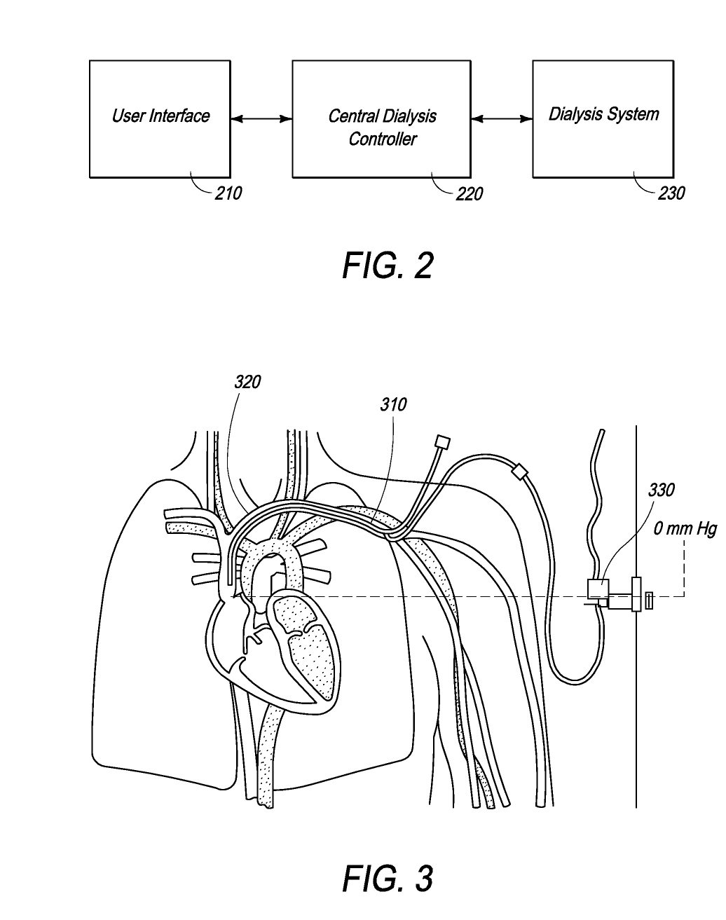

FIG. 2 is a block diagram illustrating the dialysis control system of the present invention. Referring to FIG. 2, a user interface 210 is provided that receives inputs from the user (clinician) indicating the preferred frequency of CVP measurement and the preferred range of CVP values. These inputs are supplied to the central dialysis controller 220. The central dialysis controller 220 is a programmable system that can be used to regulate CVP monitoring, and the rate of hemodialysis/ultrafiltration based on the monitored CVP. Depending on the frequency of CVP measurement determined by the user, the central dialysis controller 220 communicates a signal to the blood pump in the dialysis system 230 to stop the blood flow whenever a CVP measurement is to be recorded. Following this, a CVP sensor in the dialysis system 230 takes the measurement and communicates it to the central dialysis controller 220, which may transmit it to the user interface 210 for display. After a CVP measurement is complete, the central dialysis controller 220 communicates another signal to the dialysis system 230, causing the blood flow to resume. The central dialysis controller 220 also keeps track of the measured CVP values to determine if they are in the user-defined range. A decrease in CVP below the defined range would indicate hypovolaemia. In such a case, the central dialysis controller 220 halts the process of ultrafiltration, so that no additional fluid can be removed until CVP is restored to the desired range. In one embodiment the central dialysis controller 220 titrates the ultrafiltrate removal to the range of 2-6 mmHg, which keeps the CVP in the desired range.

The present invention contemplates a wide range of CVP measurement systems, integrated with conventional dialysis machines. Measuring CVP can be accomplished in a number of ways. In one embodiment, CVP may be measured with a sensor located at the tip of an appropriate catheter. In another embodiment, CVP may be measured with a dedicated pressure transducer located remote from the catheter, with the transducer being held at the same level as the heart. FIG. 3 is an exemplary illustration of the latter embodiment. Referring to FIG. 3, a catheter 310 used for accessing blood is shown. The catheter 310 is placed in the Central Vena Cava 320. The pressure transducer 330 measures the central venous pressure at the heart level. The CVP measurement in this case is used to control the rate of hemofiltration in the same manner as when a CVC is used.

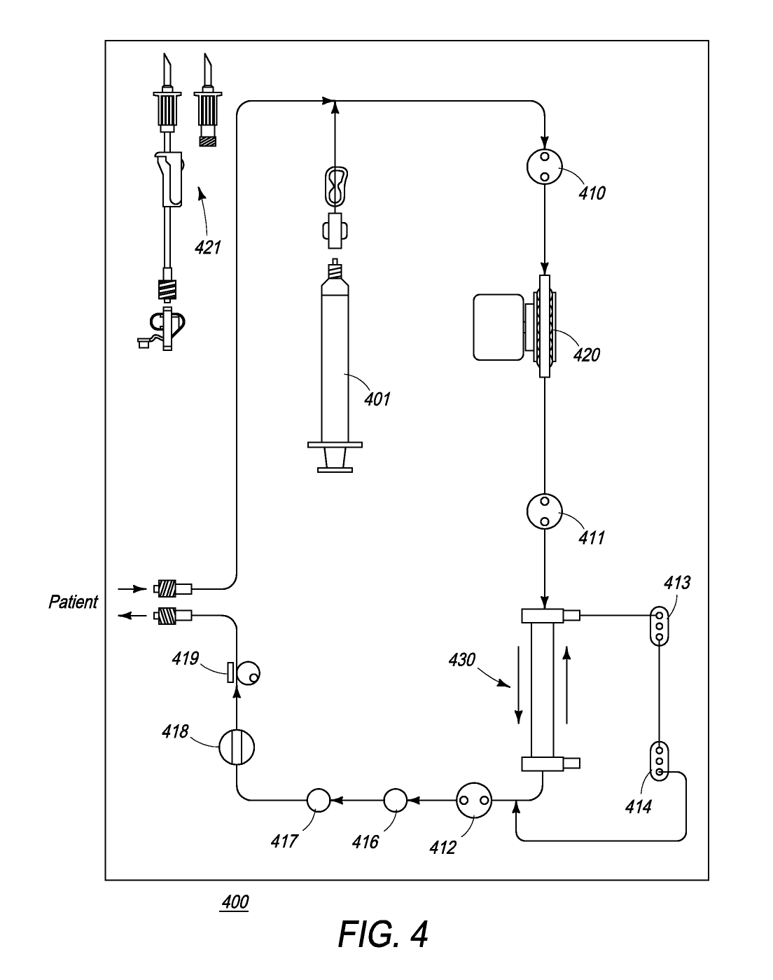

In another embodiment, CVP is measured with a remote sensor inside the hemofiltration machine. Referring to FIG. 4, an exemplary blood circuit 400 with the provision of CVP measurement is illustrated. As blood enters into the circuit 400 from the patient, an anticoagulant is injected into the blood using the syringe 401, to prevent coagulation. A pressure sensor, PBIP 410 is provided, which is used for the measurement of central venous pressure. A blood pump 420 forces the blood from the patient into the dialyzer 430. Two other pressure sensors, PBI 411 and PBO 412, are provided at the inlet and the outlet respectively of the dialyzer 430. The pressure sensors PBI 411 and PBO 412 help keep track of and maintain fluid pressure at vantage points in the hemodialysis system. A pair of bypass valves B 413 and A 414 is also provided with the dialyzer, which ensures that fluid flow is in the desired direction in the closed loop dialysis circuit. The user can remove air at the port 417 if air bubbles have been detected by sensor 418. A blood temperature sensor 416 is provided prior to the air elimination port 417. An AIL/PAD sensor 418 and a pinch valve 419 are employed in the circuit to ensure a smooth and unobstructed flow of clean blood to the patient. A priming set 421 is pre-attached to the haemodialysis system that helps prepare the system before it is used for dialysis.

For taking CVP measurement, blood flow in the circuit 400 is stopped by stopping the blood pump 420. At this point, the pressure in the catheter used for accessing blood (not shown) will equilibrate, and the pressure measured at pressure sensor PBIP 410 in the hemofiltration machine will be equal to the pressure at the catheter tip. This measured pressure (CVP) is then used to regulate the rate of ultrafiltration and the volume of fluid removed from the patient.

Thus, operationally, the system of present invention modifies a conventional dialysis system such that ultrafiltration is conducted at a rate preset by the physician. Periodically, the blood flow is stopped and the average CVP is measured, using one of the various measurement methods described above. In one embodiment, a safety mode is provided, wherein if CVP drops below a preset limit, hemofiltration is discontinued and an alarm sounded.

In another application, a hypervolemic patient such as a patient with Congestive Heart Failure (CHF) may be given ultrafiltration to remove fluids. It is known in the art that while the ultrafiltration process removes fluid from the blood, the fluid that is intended to be removed is located in the interstitial spaces. Further, the rate of fluid flow from the interstitial spaces into the blood is unknown. Without the system of present invention, a physician can only guess at the interstitial fluid removal rate that will balance fluid removal from the blood stream with the fluid flow back into the blood from the interstitial space, and sets the dialysis machine for that rate. In such a scenario, constant monitoring on the part of the physician is required to make sure that the fluid removal rate does not over or under hydrate the patient. With the system of present invention, a physician can pre-set the total amount of fluid he wants removed—typically computed from patient weight, and the minimal average CVP allowed. The system then removes fluid at the maximum rate that automatically maintains the desired CVP. That is, the system of present invention automatically balances the fluid removal rate with the fluid flow rate from the interstitial spaces into the blood.

It should be appreciated that normal CVP levels is between 2 and 6 mmHg. Elevated CVP is indicative of over hydration, while decreased CVP indicates hypovolemia. Using the present invention, a patient may being a ultrafiltration session with a CVP above normal, e.g. 7-8 mmHg, and end the session at a final CVP target of 3 mmHg through, for example, a 6 hour treatment session. However, if midway through the treatment session, CVP has fallen more than 50% of the desired drop, while the fluid removed has only reached 50% of the final target for removal, the system can be reprogrammed to reduce the goal for fluid removal or reduce the rate of fluid removal. Other actions can be taken based on more complicated algorithms. The net result is that hypovolemia is avoided by monitoring the rate and actual value of CVP.

One of ordinary skill in the art would appreciate that the present invention may also be useful in controlling fluid removal rates not only during hemofiltration, but for all types of renal replacement therapies.

While there has been illustrated and described what is at present considered to be a preferred embodiment of the present invention, it will be understood by those skilled in the art that various changes and modifications may be made, and equivalents may be substituted for elements thereof without departing from the true scope of the invention. In addition, many modifications may be made to adapt a particular situation or material to the teachings of the invention without departing from the central scope thereof. Therefore, it is intended that this invention not be limited to the particular embodiment disclosed as the best mode contemplated for carrying out the invention, but that the invention will include all embodiments falling within the scope of the appended claims.

We claim:

1. A method for regulating the volume of fluid removed from a patient during renal dialysis, the method comprising:
periodically measuring the average central venous pressure in the ventral venous line used for dialysis; and
adjusting the rate of ultrafiltration based on the measured values of central venous pressure.

2. The method of claim 1, which includes presetting the frequency of central venous pressure measurement and an acceptable range of central venous pressure values.

3. The method of claim 2, further comprising the step of discontinuing ultrafiltration when central venous pressure drops below a preset limit.

4. The method of claim 1, which includes presetting the rate of ultrafiltration.

5. The method of claim 1, which includes presetting the total volume of fluid to be removed from said patient.

6. The method of claim 1, further comprising the step of stopping the flow of blood when central venous pressure is measured.

7. The method of claim 1, wherein said method is used with any one of a hemofiltration system, a hemodiafiltration system, or a hemodialysis system.

8. A system for regulating the volume of fluid removed from a patient during renal dialysis, the system comprising:
a sensor for periodically measuring the average central venous pressure in the ventral venous line used for dialysis; and
a controller for causing said sensor to periodically measure the average central venous pressure and adjusting the rate of ultrafiltration based on the measured values of central venous pressure.

9. The system of claim 8, wherein the controller is programmable to operate according to a preset frequency of central venous pressure measurement and a preset acceptable range of central venous pressure values.

10. The system of claim 8, wherein the controller is programmable to operate according to a preset rate of ultrafiltration.

11. The system of claim 8, wherein the controller is programmable to operate according to a preset total volume of fluid that is to be removed from said patient.

12. The system of claim 8, wherein the controller is configured to discontinue ultrafiltration when central venous pressure drops below a preset limit.

13. The system of claim 8, wherein the controller stops the flow of blood when central venous pressure is measured.

14. The system of claim 8, wherein said sensor for measuring central venous pressure is located at the tip of a catheter used for accessing blood during dialysis.

15. The system of claim 8, wherein said sensor for measuring central venous pressure is located remote from the catheter used for accessing blood during dialysis.

16. The system of claim 15, wherein said sensor for measuring central venous pressure is located at the same level as the heart.

17. The system of claim 15, wherein said sensor for measuring central venous pressure is located inside the dialysis machine.

18. The system of claims 14 and 15, wherein said catheter used for accessing blood during dialysis is a central venous catheter.

19. The system of claim 18, wherein said central venous catheter is a double lumen catheter.

20. The system of claim 8, wherein said system is used with any one of a hemofiltration system, a hemodiafiltration system, or a hemodialysis system.
